(19) United States
(12) Patent Application Publication
(10) Pub. No.: US 2025/0009269 A1
(43) Pub. Date: Jan. 9, 2025

(54) STATE ESTIMATION APPARATUS, STATE ESTIMATION METHOD, AND PROGRAM

(71) Applicant: NIPPON TELEGRAPH AND TELEPHONE CORPORATION, Tokyo (JP)

(72) Inventors: Ryota NISHIZONO, Tokyo (JP); Naoki SAIJO, Tokyo (JP); Makio KASHINO, Tokyo (JP)

(73) Assignee: NIPPON TELEGRAPH AND TELEPHONE CORPORATION, Tokyo (JP)

(21) Appl. No.: 18/710,929

(22) PCT Filed: Nov. 22, 2021
(86) PCT No.: PCT/JP2021/042756

§ 371 Date: May 16, 2024

(51) Int. Cl. A61B 5/16; A61B 5/00

(52) U.S. Cl. CPC A61B 5/165; A61B 5/163; A61B 5/7275

(57) ABSTRACT

Provided is technique for estimating a psychological/cognitive state of a subject during task execution by using time-series data related to a blinking of the subject during the task execution. A time-series data acquisition unit that acquires time-series data (hereinafter referred to as subject time-series data) related to the blinking of the subject during a predetermined task, and a state estimation unit that generates a state estimation result indicating a psychological/cognitive state of the subject from the subject time-series data and time-series data (hereinafter referred to as reference time-series data) related to a blinking serving as a reference are included.

### TECHNICAL FIELD

The present invention relates to technique for estimating psychological/cognitive state of a subject during task execution.

### BACKGROUND ART

It is known that a frequency of blinking (also referred to as eyeblink) varies depending on emotion of a person who performs blinking or a task that the person performs (see NPL 1). For example, the frequency of blinking increases due to emotion of anger, and the frequency of blinking decreases due to task of reading.

### CITATION LIST

### Non Patent Literature

[NPL 1] John A. Stern, Larry C. Walrath, and Robert Goldstein, “The endogenous eyeblink”, Psychophysiology, Volume 21, Issue 1, pp. 22-33, 1984.

### SUMMARY OF INVENTION

### Technical Problem

We consider quantitative evaluation of the performance of exercise by athlete. For example, in a competition such as a car race to compete for speed, there are conventionally known indexes related to the exercise of the athlete such as time and speed. On the other hand, the indexes related to the psychological state and cognitive state of the athlete such as attention state, concentration state, and excitation state are not present, although knowledge such as that disclosed in NPL 1 is present.

Therefore, an object of the present invention is to provide technique for estimating the psychological/cognitive state of the subject during the task execution by using time-series data related to the blinking of the subject during the task execution.

### Solution to Problem

An aspect of the present invention includes a time-series data acquisition unit that acquires time-series data (hereinafter referred to as subject time-series data) related to a blinking of a subject during a predetermined task, and a state estimation unit that generates a state estimation result indicating a psychological/cognitive state of the subject from the subject time-series data and time-series data (hereinafter referred to as reference time-series data) related to a blinking serving as a reference.

### Advantageous Effects of Invention

According to the present invention, it is possible to estimate the psychological/cognitive state of the subject during the task execution.

### BRIEF DESCRIPTION OF DRAWINGS

FIG. 1 is a diagram showing time-series data that is an experiment result.

FIG. 2 is a diagram showing appearance of synchronization of blinking.

FIG. 3 is a diagram showing time-series data that is the experiment result.

FIG. 4 is a diagram showing relationship between speed of lap and intensity of synchronization.

FIG. 5 is a block diagram showing a configuration of a state estimation device 100.

FIG. 6 is a flowchart showing an operation of the state estimation device 100.

FIG. 7 is a block diagram showing a configuration of a state estimation unit 120.

FIG. 8 is a flowchart showing an operation of the state estimation unit 120.

FIG. 9 is a diagram showing an example of a functional configuration of a computer that realizes each device according to an embodiment of the present invention.

### DESCRIPTION OF EMBODIMENTS

The following describes an embodiment of the present invention in detail. Note that constituent units that have the same functions are denoted by the same numerals and repeated description thereof will be omitted.

A notation method used in this specification will be described before the embodiments are described.
\hat{ }(caret) denotes superscript. For example, x^{y\hat{ }z} means that y^{z} is a superscript to x and x_{y\hat{ }z} means that y^{z} is a subscript to
x. In addition, _ (underscore) denotes subscript. For example, x^{y_z} means that y_{z} is a superscript to x and x_{y_z} means that y_{z} is a subscript to x.

Superscripts “\hat{ }” and “˜” as in \hat{ }x and ˜x for a certain character x would normally be written directly above “x”, but are written as \hat{ }x or ˜x here due to restrictions on notation in this specification.

### TECHNICAL BACKGROUND

An embodiment of the present invention is based on newly obtained knowledge that (1) the time-series data of blinking during a predetermined task (for example, racing car traveling) shows a similar tendency between the same test subjects or between different test subjects and (2) the degree of synchronization between the time-series data of blinking is different due to a difference in psychological/cognitive load, and is to estimate the psychological/cognitive state of the subject during the task execution by using the relevant knowledge.

Experiment that serves as a basis and result thereof will be described below.

### [Experiment]

The blinking of driver of formula car which is going around racing course is measured, and the time-series data related to the blinking of driver is compared.

### [Experiment Result]

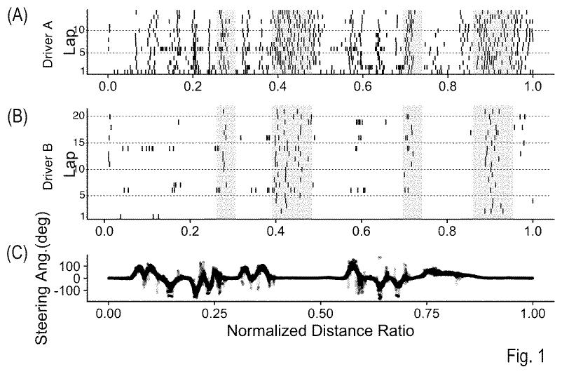

FIG. 1 is a diagram showing the time-series data that is an experiment result. FIG. 1 shows two kinds of graphs such as a graph created on the basis of a moving image obtained by photographing the blinking of driver and a graph created on the basis of the data of steering angle. The graphs of (A) and (B) are graphs showing appearance of the blinking of each driver in each lap, and the graph of (C) is a graph showing appearance of the steering of two drivers. The horizontal axis of the three graphs represents normalized distance (Normalized Distance Ratio) indicating the distance from the start point when the distance from the start point to the goal point is set to be 1. In addition, the vertical axes of the graphs (A) and (B) represent the number of laps, and the small vertical lines of the graphs (A) and (B) represent timing of the blinking of driver. On the other hand, the vertical axis of the graph (C) represents the steering angle. When the two graphs of (A) and (B) are compared, it can be seen that although the frequency of blinking is greatly different between the driver A and the driver B, there is a location where the two drivers perform blinking in common (a shaded place in the graphs of (A) and (B)). In addition, when compared together with the graph (C), it can be also seen that the frequency of the blinking of driver decreases at a corner of the racing course, and that the blinking of driver increases at the beginning of straight line of the racing course.

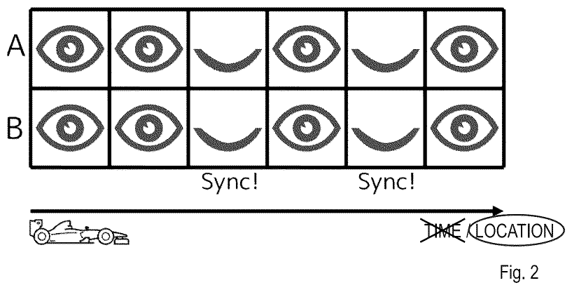

That is, it can be seen that, when the position on the racing course is used as a reference, a similar eyeblink action occurs among one driver, and a similar eyeblink action occurs between two drivers (see FIG. 2). Hereinafter, such a phenomenon will be referred to as a synchronization phenomenon of eyeblink action. The synchronization phenomenon of eyeblink action suggests that the driver is not always in a constant flat psychological/cognitive state when going around the racing course, but dynamically controls the psychological/cognitive state in accordance with the position of the racing course.

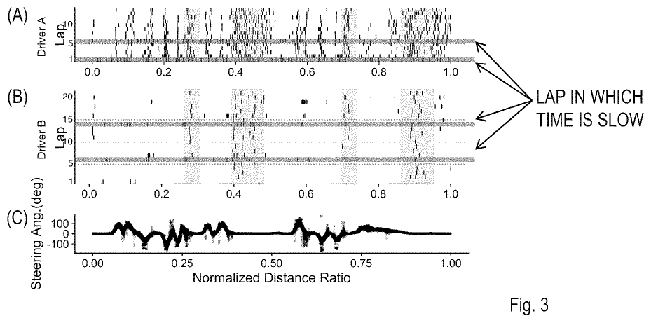

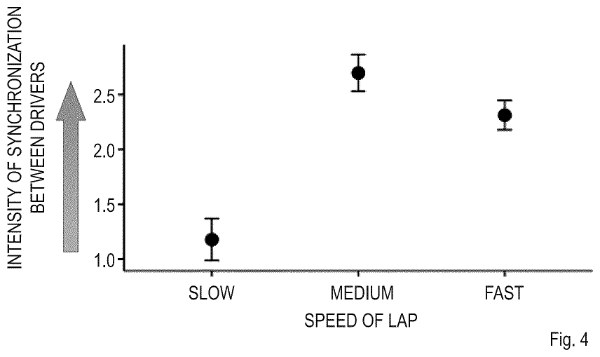

In addition, it can be also seen that the intensity of synchronization changes depending on the speed of going around by defining an index representing the intensity of synchronization for performing the eyeblink at the common location in the racing course and analyzing the time-series data shown in FIG. 1. The description will be performed below. It can be seen that the graphs of FIG. 1 (A) and FIG. 1 (B) show that there is a lap in which the blinking sparsely occurs (see FIG. 3). The lap in which the blinking sparsely occurs is a lap in which time is slow. Therefore, when the relationship with the intensity of synchronization is examined by dividing the laps into laps in which time is slow, laps in which time is medium and laps in which time is fast, the relationship with the intensity of synchronization is shown in FIG. 4. FIG. 4 shows the intensity of synchronization at laps in which time is slow, laps in which time is medium, and laps in which time is fast between the driver A and the driver B, and it can be seen that the intensity of synchronization is weak at the laps in which time is slow, while strong synchronization appears at the laps in which time is medium or fast. Since the speed of lap is considered to be strongly related to the degree of difficulty for the driver, it is considered that the degree of concentration, which is a psychological/cognitive load, can be expressed by an index of the degree of synchronization.

From the above-described experiment result, it can be seen that the above-described knowledge of (1) and (2) are obtained.

### First Embodiment

A state estimation device 100 estimates the psychological/cognitive state of the subject during the task execution on the basis of time-series data (hereinafter referred to as subject time-series data) related to the blinking of the subject during the predetermined task. Here, the predetermined task is a task for going around a certain specific course or a task for performing a specific operation at predetermined timing. As an example of the task for going around the specific course, there is a car race in which racing cars are going around, a bicycle race in which bicycles are going around, a boat race in which boats are going around, and a running race in which persons are going around by his or her own foot. In addition, as an example of the task for performing the specific operation at the predetermined timing, there is a batting for swinging a bat at timing of pitching by a pitcher and a base stealing for traveling from first base to second base at timing of pitching by the pitcher in baseball, an operation for pulling a bow to hit a target in archery, and an operation for throwing a ball in shot put.

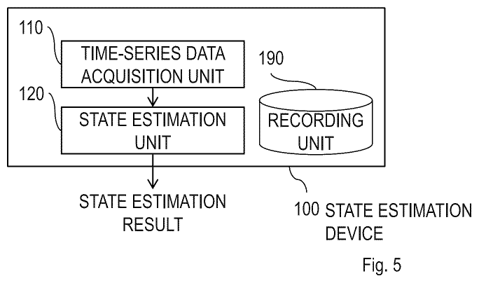

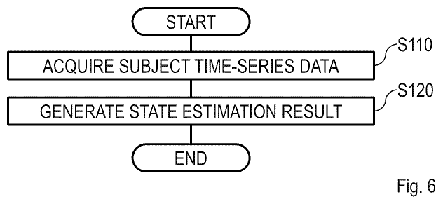

The state estimation device 100 will be described below with reference to FIG. 5 and FIG. 6. FIG. 5 is a block diagram showing a configuration of the state estimation device 100. FIG. 6 is a flowchart showing an operation of the state estimation device 100. As shown in FIG. 5, the state estimation device 100 includes a time-series data acquisition unit 110, a state estimation unit 120, and a recording unit 190. The recording unit 190 is a constituent unit that appropriately records information necessary for processing of the state estimation device 100. The recording unit 190 previously records time-series data (hereinafter referred to as reference time-series data) related to a blinking serving as a reference. Here, the reference time-series data is data used for estimating a psychological/cognitive state of subject, and is time-series data related to a blinking in the same task as the task of the subject time-series data. The reference time-series data may be data obtained by performing a task by the subject himself or herself in advance, or may be data obtained by performing a task by one or more persons different from the subject in advance.

An operation of the state estimation device 100 will be described with reference to FIG. 6.

In a step S110, the time-series data acquisition unit 110 acquires the subject time-series data. The time-series data acquisition unit 110 can acquire, for example, the subject time-series data from a moving image obtained by photographing the blinking of the subject during the task execution.

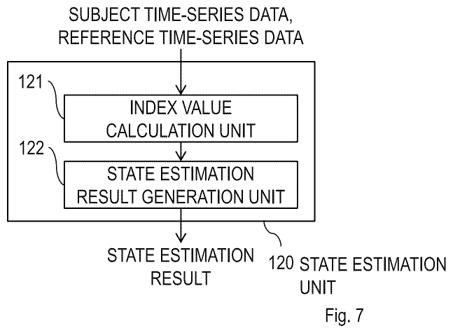

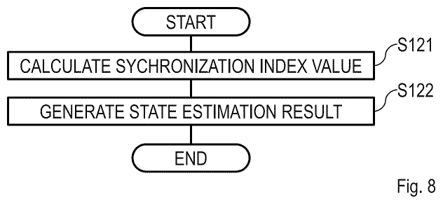

In a step S120, the state estimation unit 120 generates a state estimation result indicating the psychological/cognitive state of the subject from the subject time-series data acquired in the step S110 and the reference time-series data recorded in the recording unit 190. The state estimation unit 120 will be described below with reference to FIG. 7 and FIG. 8. FIG. 7 is a block diagram showing a configuration of the state estimation unit 120. FIG. 8 is a flowchart showing an operation of the state estimation unit 120. As shown in FIG. 7, the state estimation unit 120 includes an index value calculation unit 121 and a state estimation result generation unit 122.

An operation of the state estimation unit 120 will be described with reference to FIG. 8.

In a step S121, the index value calculation unit 121 calculates an index value (hereinafter referred to as a synchronization index value) indicating a degree of synchronization between the subject time-series data and the reference time-series data from the subject time-series data and the reference time-series data. As the synchronization index value, z value can be used, for example.

In a step S122, the state estimation result generation unit 122 generates a state estimation result from the synchronization index value calculated in the step S121. For example, when the state estimation result indicates the degree of concentration of the subject, the state estimation result indicates that the degree of concentration of the subject is higher as the synchronization index value is higher.

According to the embodiment of the present invention, it is possible to estimate the psychological/cognitive state of the subject during the task execution.

### <Supplementary Note>

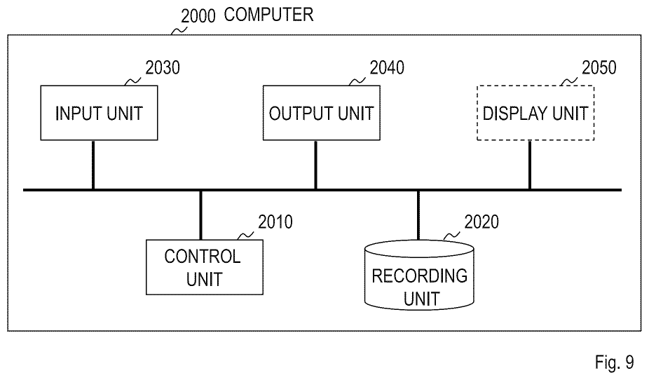

FIG. 9 is a diagram showing an example of a functional configuration of a computer 2000 that realizes each of the above-described devices. The processing in each of the above-described devices can be performed by causing a recording unit 2020 to read a program for causing the computer 2000 to function as each of the above-described devices, and causing a control unit 2010, an input unit 2030, an output unit 2040, and the like to operate.

The device of the present invention includes, for example, as single hardware entity, an input unit to which a keyboard or the like can be connected, an output unit to which a liquid crystal display or the like can be connected, a communication unit to which a communication device (for example, a communication cable) capable of communication with the outside of the hardware entity can be connected, a CPU (Central Processing Unit, which may include a cache memory, a register, and the like), a RAM or a ROM that is a memory, an external storage device that is a hard disk, and a bus for connecting the input unit, the output unit, the communication unit, the CPU, the RAM, the ROM, and the external storage device so as to allow data communication therebetween. Also, if necessary, the hardware entity may be provided with a device (drive) or the like capable of reading and writing data from/to a recording medium such as a CD-ROM. Examples of a physical entity including such hardware resources include a general-purpose computer.

Programs required to implement the above-described functions, data required to process the program, and the like are stored in the external storage device of the hardware entity (the storage device is not limited to the external storage device and, for example, the program may be stored in the ROM which is a read-only storage device). In addition, data or the like obtained by processing of these programs is appropriately stored in the RAM, the external storage device, or the like.

In the hardware entity, each program stored in the external storage device (or a ROM or the like) and data necessary for processing of each program are read to the memory as necessary, and interpreted, executed, and processed by the CPU as appropriate. As a result, the CPU implements predetermined functions (each constituent unit represented above as unit, means, and the like).

The present invention is not limited to the above-described embodiment, and appropriate changes can be made without departing from the spirit of the present invention. In addition, the processing described in the embodiments is not only executed in time-series in the described order, but also may be executed in parallel or individually in accordance with processing capability of a device that executes the processing or as necessary.

As described above, when the processing function in the hardware entity (the device according to the present invention) described in the above-described embodiments is implemented by the computer, processing content of the function included in the hardware entity is written by the program. Then, by executing this program on the computer, the processing function in the above-described hardware entity is implemented on the computer.

The program writing this processing content can be recorded on a computer-readable recording medium. An example of the computer-readable recording medium may include any recording medium such as a magnetic recording device, an optical disc, a magneto-optical recording medium, and a semiconductor memory.

Specifically, for example, a hard disk device, a flexible disk, a magnetic tape, or the like can be used as the magnetic recording device, a DVD (Digital Versatile Disc), a DVD-RAM (Random Access Memory), a CD-ROM (Compact Disc Read Only Memory), a CD-R (Recordable)/RW (ReWritable), or the like can be used as the optical disk, an MO (Magneto-Optical disc) or the like can be used as the magneto-optical recording medium, and an EEP-ROM (Electronically Erasable and Programmable-Read Only Memory) or the like can be used as the semiconductor memory.

In addition, the program is distributed, for example, by sales, transfer, lending, or the like of a portable recording medium such as the DVD or the CD-ROM on which the program is recorded. Further, the distribution of program may be performed by storing the program in a storage device of a server computer and transferring the program from the server computer to another computer via a network.

The computer that executes such a program first temporarily stores, for example, the program recorded on the portable recording medium or the program transferred from the server computer in the own storage device. Then, when processing is executed, the computer reads the program stored in the own storage device and executes the processing in accordance with the read program. In addition, as another form of execution of the program, the computer may directly read the program from the portable recording medium and execute the processing in accordance with the program, and further, processing in accordance with the received program may be sequentially executed each time the program is transferred from the server computer to the computer. Furthermore, instead of transferring the program from the server computer to the computer, the processing described above may be executed by a so-called ASP (Application Service Provider) type service, in which a processing function is realized by execution instructions and result acquisition alone. Note that the program according to the present embodiment includes information to be used for processing by an electronic computer and equivalent to the program (data which is not a direct command to the computer but has a property that regulates the processing of the computer and the like).

In addition, although the hardware entity is configured by executing a predetermined program on the computer in the embodiment, at least a part of the processing content of the hardware entity may be realized in hardware.

The above description of the embodiments of the present invention is presented for the purpose of illustration and description. There is no intention to be exhaustive and there is no intention to limit the invention to a disclosed exact form. Modifications or variations are possible from the above-described teachings. The embodiments are selected and expressed in order to provide the best illustration of the principle of the present invention and to enable those skilled in the art to utilize the present invention in various embodiments and with various modifications so that the present invention is suitable for their contemplated actual use. All of such modifications or variations are within the scope of the present invention defined by the appended claims interpreted in accordance with a range given fairly, legally and impartially.

1. A state estimation device comprising:
a time-series data acquisition circuitry configured to acquire time-series data (hereinafter referred to as subject time-series data) related to a blinking of a subject during a predetermined task; and
a state estimation circuitry configured to generate a state estimation result indicating a psychological/cognitive state of the subject from the subject time-series data and time-series data (hereinafter referred to as reference time-series data) related to a blinking serving as a reference.

2. The state estimation device according to claim 1, wherein
the state estimation circuitry comprises
an index value calculation circuitry that calculates an index value (hereinafter referred to as a synchronization index value) indicating a degree of synchronization between the subject time-series data and the reference time-series data from the subject time-series data and the reference time-series data, and
a state estimation result generation circuitry that generates the state estimation result from the synchronization index value.

3. The state estimation device according to claim 2, wherein
the state estimation result indicates a degree of concentration of the subject, and indicates that the degree of concentration of the subject is higher as the synchronization index value is higher.

4. The state estimation device according to claim 1, wherein
the predetermined task is a task for going around a certain specific course or a task for performing a specific operation at predetermined timing.

5. A state estimation method comprising:
a time-series data acquisition step of acquiring time-series data (hereinafter referred to as subject time-series data) related to a blinking of a subject during a predetermined task by a state estimation device; and
a state estimation step of generating a state estimation result indicating a psychological/cognitive state of the subject from the subject time-series data and time-series data (hereinafter referred to as reference time-series data) related to a blinking serving as a reference by the state estimation device.

6. A non-transitory computer-readable storage medium which stores a program for causing a computer to function as the state estimation device according to claim 1.
